(19) United States
(12) Patent Application Publication
(10) Pub. No.: US 2003/0091509 A1
(43) Pub. Date: May 15, 2003

(54) Novel use of pulmonary surfactant for the prophylaxis and treatment of chronic pulmonary diseases

(76) Inventors: Dietrich Häfner, (US); Andreas Keller, Reichenau (DE); Frank Rathgeb, Konstanz (DE); Peter Schaffer, Radolfzell (DE); Wilhelm Wurst, Konstanz (DE); Christoph Karl, Konstanz (DE)

Correspondence Address: NATH & ASSOCIATES, 1030 15th STREET, 6TH FLOOR, WASHINGTON, DC, 20005, US

(21) Appl. No.: 10/182,621

(22) PCT Filed: Feb. 10, 2001
(86) PCT No.: PCT/EP01/01485

(30) Foreign Application Priority Data
Feb. 11, 2000 (EP) 00102858.8

(51) Int. Cl. A61K 38/17; A61L 9/04

(52) U.S. Cl. 424/45; 514/2

(57) ABSTRACT

The invention describes the novel use of pulmonary surfactant preparations for the prophylaxis or treatment of chronic pulmonary diseases in mammals.

### TECHNICAL FIELD OF THE INVENTION

The invention relates to the novel use of pulmonary surfactant preparations for the prophylaxis or treatment of chronic pulmonary diseases.

### PRIOR ART

Nowadays various therapies are used for the long-term treatment of chronic pulmonary diseases, for example pharmacological therapies of asthma by beta-2 sympathomimetics, corticosteroids, parasympatholytics, theophylline or alternatively antiallergics or the therapy of pulmonary emphysema with corticosteroids or by oxygen long-term therapy. Chronic pulmonary diseases are often associated with dyspnea. This dyspnea can also occur episodically, in particular in the case of asthma and COPD. Such exacerbations then have to be treated by the administration of corticosteroids or alternatively using invasive or noninvasive ventilation of the patient, e.g. with oxygen. In this case, inpatient admission of the patient in hospital is often unavoidable, such as in the case of acute COPD exacerbations [Kessler at al.: Predictive Factors of Hospitalization for Acute Exacerbation in a Series of 64 Patients with Chronic Obstructive Pulmonary Disease, Am J Respir Crit Care Med, Vol 159. pp 158-164, 1999]. Such an emergency treatment, however, also involves a risk of side effects, e.g. damage to the lungs can occur due to mechanical ventilation of the patient and the weaning of the patient from the ventilation often turns out to be problematical [T. Welte et al., Weaning of Patients with Respiratory Failure due to COPD: Noninvasive (Face Mask) versus Invasive (Endotracheal Tube) Ventilation, Eur Respir Top 1999; 5: 13].

In addition to the abovementioned standard treatments, more recently alternative therapeutic approaches have been followed for the treatment of chronic pulmonary diseases. Thus Kurashima et al. [A Pilot Study of Surfactant Inhalation for the Treatment of Asthmatic Attack, Jpn. J. Allergol. 40 (2), 160-163, 1991] describe the results of a pilot study on the administration of the pulmonary surfactant preparation Surfacten® in patients having an asthmatic attack. Oetomo et al. (Surfactant Nebulization Does not Alter Airflow Obstruction and Bronchial Responsiveness to Histamine in Asthmatic Children, Am. J. Respir Crit Care Med 1996; 153; 1148-1152) report that the administration of surfactant does not show any positive action in children with asthma. Wirtz et al. (Exogenous Surfactant Application in Respiratory Failure due to Chronic Obstructive Pulmonary Disease, Respiration 1995; 62:157-159) describe the administration of exogenous surfactant (Survanta®) in a case of respiratory failure as a result of a chronic disorder of the bronchopulmonary system with obstructive ventilation disorder. Lusardi et al. [Role of Surfactant in Chronic Obstructive Pulmonary Disease: Therapeutic Implications, Respiration 1992; 59 (suppl 1):28-32] question the value of such a therapy for increasing surfactant in COPD. Anzueto et al. (Effects of Aerosolized Surfactant in Patients With Stable Chronic Bronchitis, JAMA, 1997, Vol 278, No. 17, 1426-1431) describe the effects of surfactant in patients with stable chronic bronchitis. Griese et al. (Nebulisation of a Bovine Surfactant in Cystic Fibrosis: a Pilot Study, Eur Respir J 1997; 10: 1989-1994) describe a pilot study for the administration of surfactant (Alveofact®) in patients with cystic fibrosis. In this study, no acute or short-term positive effects were observed in young adults with cystic fibrosis.

### DESCRIPTION OF THE INVENTION

The object of the present invention is the provision of alternative treatment methods and medicaments for the prophylaxis or treatment of chronic pulmonary diseases in mammals. Surprisingly, it has now been found that pulmonary surfactant preparations, in particular those which contain recombinantly prepared pulmonary surfactant proteins, are suitable for the prophylaxis or treatment of chronic pulmonary diseases in mammals. In particular, pulmonary surfactant can also be employed in the sense of an emergency treatment in an acute exacerbation in the course of chronic pulmonary disease. Treatment with pulmonary surfactant is characterized by good tolerability and rapid efficacy. The side effects of conventional emergency treatments can be decreased or avoided and ventilation of the patient as well as inpatient treatment in hospital can be shortened or completely avoided.

In a first aspect, the invention therefore relates to the use of a pulmonary surfactant preparation for the production of medicaments for the prophylaxis or treatment of chronic pulmonary diseases.

Chronic pulmonary diseases in the sense of the invention are in particular diseases of the type of chronic diseases of the bronchopulmonary system with obstructive ventilation disorders (chronic obstructive pulmonary disease, also called COPD below), asthma, cystic fibrosis, pulmonary fibrosis, pulmonary degeneration, chronic bronchitis and pulmonary emphysema.

According to the invention, prophylaxis or treatment of chronic pulmonary diseases is understood as meaning, in particular, also the prophylaxis or treatment of an exacerbation, in particular of an acute exacerbation, in the course of a chronic pulmonary disease, for example an acute asthma attack. Exacerbation of a chronic pulmonary disease is understood according to the invention as meaning, in particular, the progression, intensification or the breaking out again of one of the chronic pulmonary diseases according to the invention, where the exacerbation can comprise, in particular, a worsening of the pulmonary function. For example, in the case of asthma mention may be made of exacerbations which are caused by physical stress, infection-related exacerbations, exacerbations caused by inhalation of allergens, exacerbations caused by inhalation of cold air or toxic substances and acute COPD exacerbations.

According to the invention, the mammals are preferably humans.

Natural pulmonary surfactant has surface-active properties; it reduces, for example, the surface tension in the alveoli. A simple and rapid in vitro test with which the surface activity of pulmonary surfactant can be determined is, for example, the so-called Willhelmy balance [Goerke, J. Biochim. Biophys. Acta, 344: 241-261 (1974), King R. J. and Clements J. A., Am. J. Physicol. 223: 715-726 (1972)]. This method gives information on the pulmonary surfactant quality, measured as the action of a pulmonary surfactant of achieving a surface tension of almost zero mN/m. Another measuring device for determining the surface activity of pulmonary surfactant is the pulsating bubble surfactometer [Possmayer F., Yu S. and Weber M., Prog. Resp. Res., Ed. v. Wichert, Vol. 18: 112-120 (1984)].

The activity of a pulmonary surfactant preparation can also be determined by means of in vivo tests, for example as described by Häfner et al. (D. Häfner et al.: Effects of rSP-C surfactant on oxygenation and histology in a rat lung lavage model of acute lung injury. Am. J. Respir. Crit. Care Med. 1998, 158: 270-278). By the measurement of, for example, the pulmonary compliance, the blood gas exchange or the ventilation pressures needed, it is possible to obtain information on the activity of a pulmonary surfactant.

Pulmonary surfactant preparation is understood according to the invention as meaning the numerous known compositions and their modifications which have the function of natural pulmonary surfactant. In this case, preferred compositions are those which, for example, have activity in the tests described above. Particularly preferred compositions are those which exhibit increased activity in such a test in comparison with natural, in particular human, pulmonary surfactant. In this context, these can be compositions which only contain phospholipids, but also compositions which, apart from the phospholipids, inter alia additionally contain pulmonary surfactant protein. Preferred phospholipids according to the invention are dipalmitoylphosphatidylcholine (DPPC), palmitoyloleylphosphatidylglycerol (POPG) and/or phosphatidylglycerol (PG). Particularly preferably, the phospholipids are mixtures of various phospholipids, in particular mixtures of dipalmitoylphosphatidylcholine (DPPC) and palmitoyloleylphosphatidylglycerol (POPG), preferably in the ratio from 7 to 3 to 3 to 7. Commercial products which may be mentioned are Curosurf® (Serono, Pharma GmbH, Unterschleiβheim) a natural surfactant from homogenized porcine lungs, Survanta® (Abbott GmbH, Wiesbaden) and Alveofact® (Boehringer Ingelheim), both extracts of bovine lungs, as well as Exosurf® (Glaxo Wellcome GmbH), a synthetic phospholipid containing excipients. Suitable pulmonary surfactant proteins are both the proteins obtained from natural sources, such as pulmonary lavage or extraction from amniotic fluid, or the proteins prepared by genetic engineering or chemical synthesis. According to the invention, in particular the pulmonary surfactant proteins designated by SP-B and SP-C and their modified derivatives are of interest. The amino acid sequences of these pulmonary surfactant proteins, their isolation or preparation by genetic engineering are known (e.g. from WO86/03408, EP-A-0 251 449, WO89/04326, WO87/06943, WO88/03170, WO91/00871, EP-A-0 368 823 and EP-A-0 348 967). Modified derivatives of the pulmonary surfactant proteins designated by SP-C, which differ from human SP-C by the replacement of a few amino acids, are described, for example, in WO91/18015 and WO95/32992. Particularly to be emphasized in this connection are the recombinant SP-C derivatives which are disclosed in WO95/32992, in particular those which differ from human SP-C in positions 4 and 5 by the replacement of cysteine by phenylalanine and in position 32 by the replacement of methionine by isoleucine [designated below as rSP-C (FF/l) or lusupultide (INN)]. Modified derivatives of pulmonary surfactant proteins are also understood as meaning those proteins which have a completely originally designed amino acid sequence with respect to their pulmonary surfactant properties, such as are described in EP-A-0 593 094 and WO 92/22315. Preferably, the polypeptide KL4 (INN: sinapultide) may be mentioned in this connection. The name pulmonary surfactant protein, according to the invention, also comprises mixtures of different pulmonary surfactant proteins. In EP-B-0 100 910, EP-A-0 110 498, EP-B-0 119 056, EP-B-0 145 005 and EP-B-0 286 011, phospholipid compositions with and without pulmonary surfactant proteins are described which are likewise suitable as components of the preparations.

As further constituents which can be present in pulmonary surfactant preparations, fatty acids such as palmitic acid may be mentioned. The pulmonary surfactant preparations can also contain electrolytes such as calcium, magnesium and/or sodium salts (for example calcium chloride, sodium chloride and/or sodium hydrogencarbonate) in order to establish an advantageous viscosity. Preferred preparations according to the invention contain 80 to 95% by weight of phospholipids, 0.5 to 3.0% by weight of pulmonary surfactant proteins, 3 to 15% by weight of fatty acid, preferably palmitic acid, and 0 to 3% by weight of calcium chloride.

The pulmonary surfactant preparations are prepared by processes known per se and familiar to the person skilled in the art, for example as described in WO95/32992. According to the invention, the pulmonary surfactant preparations are preferably lyophilized and in particular spray-dried pulmonary surfactant preparations. Lyophilized preparations are disclosed, for example, in WO 97/35882, WO 91/00871 and DE 3229179. WO 97/26863 describes a process for the preparation of powdered pulmonary surfactant preparations by spray drying. According to the invention, preparations prepared in this way are preferred.

A further subject of the invention is a method of prophylaxis or treatment of chronic pulmonary diseases in mammals. Particular mention may in this case also be made of a method of prophylaxis or treatment of exacerbations of the chronic pulmonary diseases according to the invention. The method comprises administering a therapeutically efficacious and pharmacologically tolerable amount of a pulmonary surfactant preparation to the mammal concerned. The pulmonary surfactant preparations are advantageously administered in the order of magnitude customary for pulmonary surfactant preparations.

The pulmonary surfactant preparation is administered in a manner known to the person skilled in the art, preferably by intratracheal instillation (infusion or bolus) of a pulmonary surfactant solution or suspension or in the form of an atomization of a pulmonary surfactant solution or suspension or by atomization of pulmonary surfactant powder. Preferably, the preparations according to the invention for administration are dissolved or suspended in a suitable solvent or resuspension medium, in particular if the preparations are present in lyophilized or spray-dried form. Preferably, the suitable resuspension medium is a physiological saline solution. It has proven advantageous to administer suspensions or solutions of the preparations according to the invention which contain 12.5 to 100 mg of phospholipids per ml of suspension. Preferably, the preparations according to the invention are administered per application in such an amount that the amount of phospholipids is between 12.5 and 200 mg per kilogram of body weight. As a rule, administration is carried out 1 to 3 times daily over a period of 1 to 7 days. A process is preferred in which the pulmonary surfactant solution employed contains 0.5 to 2.0 mg of rSP-C (FF/l) per ml of solvent. Particular mention may be made of a process in which the pulmonary surfactant solution employed contains 0.75 to 1.5 mg of rSP-C (FF/l) per ml of solvent. If desired, before the administration of the preparations according to the invention a bronchoalveolar lavage, preferably with dilute pulmonary surfactant preparation, can be carried out. Such a procedure is described, for example, in Gommers et al. [Bronchoalveolar lavage with a diluted surfactant suspension prior to surfactant instillation improves the effectiveness of surfactant therapy in experimental acute respiratory distress syndrome (ARDS), Intensive Care Med. 1998, 24:494-500] and in WO98/49191.

A further subject of the invention is a commercial product consisting of a customary secondary packaging, a primary packaging comprising a pharmaceutical preparation and, if desired, a pack insert, the pharmaceutical preparation being suitable for the prophylaxis or treatment of chronic pulmonary diseases in mammals and reference being made on the secondary packaging or on the pack insert of the commercial product to the suitability of the pharmaceutical preparation for the prophylaxis or treatment of chronic pulmonary diseases in mammals, and the pharmaceutical preparation being a pulmonary surfactant preparation. The secondary packaging, the primary packaging comprising the pharmaceutical preparation and the pack insert otherwise correspond to what the person skilled in the art would regard as standard for pharmaceutical preparations of this type. Suitable primary packagings are, for example, ampoules or bottles of suitable materials such as transparent polyethylene or glass or alternatively suitable means of administration such as are customarily employed for the administration of active compounds into the lungs. By way of example, mention may be made of means of administration for the atomization of an active compound solution or suspension or for the atomization of active compound powder. Preferably, the primary packaging is a glass bottle which can be sealed, for example, by a commercially available rubber stopper or a septum. A suitable secondary packaging which may be mentioned by way of example is a folding box.

A further subject of the invention are also medicaments for the prophylaxis or treatment of chronic pulmonary diseases in mammals, which contain pulmonary surfactant preparations in combination with other medicaments suitable for the treatment or prophylaxis of chronic pulmonary diseases. The pulmonary surfactant preparations according to the invention are suitable in this context, in particular, as a supplement to a long-term treatment of the patient with other medicaments for the prophylaxis or treatment of exacerbations of chronic pulmonary diseases which occur. In connection with the prophylaxis or treatment of cystic fibrosis, mention may in particular be made of the combination of pulmonary surfactant preparations with antibiotics, in particular with tobramycin.

### EXAMPLES

A.) Production of Powdered Pulmonary Surfactant Preparations

Powdered pulmonary surfactant preparations are produced by the process described in WO 97/26863:

### Example 1

7.0 g of 1,2-dipalmitoyl-3-sn-phosphatidylcholine, 2.5 g of 1-palmitoyl-2-oleoyl-3-sn-phosphatidylglycerol sodium, 205 mg of calcium chloride dihydrate and 250 mg of palmitic acid are dissolved in 300 ml of ethanol/water (85:15) with warming to 60° C., cooled to room temperature and mixed with 350 ml of a solution of rSP-C (FF/l) in chloroform/methanol 9:1 (c=429 mg/l). The resulting solution is spray-dried in a Büchi B 191 laboratory spray dryer. Spray conditions: drying gas air, inlet temperature 90° C., outlet temperature 52-54° C. A relatively loose powder is obtained.

### Example 2

A solution of the surfactant obtained from bovine lungs (obtained by extraction and purification steps such as described, for example, in EP 406732) in chloroform/methanol is spray-dried under the following conditions: Büchi B 191 laboratory spray dryer, drying gas nitrogen, inlet temperature 80° C., outlet temperature 50-52° C. A fine, yellowish powder is obtained.

### Example 3

10.95 g of 1,2-dipalmitoyl-3-sn-phosphatidylcholine, 4.6 g of 1-palmitoyl-2-oleoyl-3-sn-phosphatidylglycerol ammonium, 418 mg of calcium chloride dihydrate and 750 mg of palmitic acid are dissolved in 330 ml of 2-propanol/water (85:15) at 50° C. and, after cooling to 30° C., mixed with 620 ml of a solution of rSP-C (FF/l) in isopropanol/water (95:5, c=484 mg/l). The resulting solution is spray-dried in a Büchi B 191 laboratory spray dryer. Spray conditions: drying gas nitrogen, inlet temperature 100° C., outlet temperature 58-60° C. A colorless powder is obtained.

### Example 4

3.74 g (5.1 mmol) of 1,2-dipalmitoyl-3-sn-phosphatidylcholine, 2.81 g (3.7 mmol) of 1-palmitoyl-2-oleoyl-3-sn-phosphatidylcholine, 2.90 g (3.9 mmol) of 1,2-dipalmitoylphosphatidyl-3-sn-phosphatidylglycerol sodium, 234 mg of palmitic acid and 279 mg (1.9 mmol) of calcium chloride dihydrate are dissolved in 160 ml of 2-propanol/water (85:15) at 50° C. and, after cooling to 30° C., mixed with 566 ml of a solution of rSP-C (FF/l) in isopropanol/water (92:8, c=330 mg/l) at 30° C. The resulting solution is spray-dried in a Büchi B 191 laboratory spray dryer. Spray conditions: drying gas nitrogen, inlet temperature 90° C., outlet temperature 58-60° C. A colorless powder is obtained.

### Example 5

0.5 g of KL4 (INN: sinapultide), 7.125 g of 1,2-dipalmitoyl-3-sn-phosphatidylcholine and 2.43 g of 1-palmitoyl-2-oleoyl-3sn-phosphatidylglycerol ammonium are dissolved in 500 ml of chloroform/methanol 1:1 with warming to 45° C. and then spray-dried in a Büchi B 191 laboratory spray dryer. Spray conditions: drying gas nitrogen, inlet temperature 85° C., outlet temperature 55° C. A colorless powder is obtained.

### Example 6

A solution of phospholipids, palmitic acid and calcium chloride dihydrate obtainable according to Example 1, 3 or 4 is spray-dried—without addition of a solution of rSP-C (FF/l)—corresponding to the conditions according to Example 1, 3 or 4. A powder is obtained.

B.) Production of the Medicaments According To The Invention

### Example 1

0.1 to 10 g of the powder obtained according to Example 1 are dispensed into a bottle of volume 100 to 250 ml and the bottle is sealed. The bottle is packed in a suitable folding box together with a pack insert.

1. The use of a pulmonary surfactant preparation for the production of medicaments for the prophylaxis or treatment of chronic pulmonary diseases in mammals.

2. The use as claimed in claim 1, the mammals being humans.

3. The use as claimed in claim 1, the chronic pulmonary diseases being a disease of the type of chronic diseases of the bronchopulmonary system with obstructive ventilation disorders (COPD), asthma, cystic fibrosis, pulmonary fibrosis, pulmonary degeneration, chronic bronchitis or pulmonary emphysema.

4. The use as claimed in claim 3, the pulmonary surfactant preparation comprising phospholipids, the pulmonary surfactant proteins SP-B and/or SP-C and/or their modified derivatives, if desired together with further excipients.

5. The use as claimed in claim 4, the pulmonary surfactant protein being recombinantly prepared pulmonary surfactant protein.

6. The use as claimed in claim 5, the pulmonary surfactant protein being lusupultide.

7. The use as claimed in claim 1, the production of medicaments for the prophylaxis or treatment of exacerbations of chronic pulmonary diseases being concerned.

8. A method of prophylaxis or treatment of chronic pulmonary diseases in mammals including humans, a therapeutically efficacious and pharmacologically tolerable amount of a pulmonary surfactant preparation being administered to the sick mammal.

9. The method as claimed in claim 8, in which the pulmonary surfactant preparation comprises phospholipids and the pulmonary surfactant protein comprises lusupultide, if desired together with further excipients.

10. A commercial product, comprising a customary secondary packaging, a primary packaging comprising a pharmaceutical preparation and, if desired, a pack insert, the pharmaceutical preparation being suitable for the prophylaxis or treatment of chronic pulmonary diseases in mammals and reference being made on the secondary packaging or on the pack insert of the commercial product to the suitability of the pharmaceutical preparation for the prophylaxis or treatment of chronic pulmonary diseases in mammals, and the pharmaceutical preparation being a pulmonary surfactant preparation.
